(19) United States
(12) Patent Application Publication
(10) Pub. No.: US 2021/0391065 A1
(43) Pub. Date: Dec. 16, 2021

(54) METHOD FOR SELECTING A WOUND PRODUCT FOR A PATIENT

(71) Applicant: MÖLNLYCKE HEALTH CARE AB, GÖTEBORG (SE)

(72) Inventors: Jan BOBERG, Lerum (SE); Anand CHANDARANA, Luton Bedfordshire (GB)

(73) Assignee: MÖLNLYCKE HEALTH CARE AB, GÖTEBORG (SE)

(21) Appl. No.: 17/291,832

(22) PCT Filed: Dec. 17, 2019
(86) PCT No.: PCT/SE2019/051301

§ 371 Date: May 6, 2021

(30) Foreign Application Priority Data
Dec. 18, 2018 (SE) 1851607-0

(51) Int. Cl. G16H 40/20; G16H 10/60; G16H 50/50; G16H 50/70; G16H 50/20; G06T 7/00; G06T 17/00

(52) U.S. Cl. CPC G16H 40/20; G16H 10/60; G16H 50/50; G16H 50/70; A61B 5/107; G06T 7/0016; G06T 17/00; G06T 2207/10016; G16H 50/20

(57) ABSTRACT

The present disclosure generally relates to a computer implemented method for adaptively selecting at least one wound product for a patient, for example adapted to take into account a healing trajectory determined for a wound of the patient. The present disclosure also relates to a corresponding computer system and computer program product.

### TECHNICAL FIELD

The present disclosure generally relates to a computer implemented method for adaptively selecting a wound product for a patient, for example adapted to take into account a healing trajectory determined for a wound of the patient. The present disclosure also relates to a corresponding computer system and computer program product.

### BACKGROUND

The traditional approach when treating a wound of a patient is for a caregiver, such as a physician, to use personal experience, guidelines and best practices to select the most suitable wound products. Such wound products may for example include a wound dressing and/or a topical applicant, to be used at or in relation to the wound. Although physicians maintain a high knowledge base, they are human and sometimes may not be aware of recent development within the area, may not recognize how the healing is progressing or be aware of relevant medical information for the patient. This may in some situations result in a subjective judgement that in turn may lead to an unnecessary extension for treatment and/or a reduced healing rate for the wound.

Recently, digital solutions have been introduced for assisting the physician, greatly reducing subjectiveness in regards to the physician's decision making. However, such digital solutions are prone to provide generalized recommendation, not allowing for an adequate level of adaptation, which for example may be necessary depending on the clinic and the availability of different type of wound products.

US2009216553 present an example of such a digital solution. However, US2009216553 provides further limitations to the physician's decision making, only providing recommendations where a wound product is reimbursable and provided by an insurance provider.

With the above in mind, there appears to be room for further improvements, focusing on balancing the outcome from a digital decision-making solution with the need to allow the physician to take into account parameters not readily available for such a digital decision-making solution.

### SUMMARY

In view of above-mentioned and other drawbacks of the prior art, it is an object of the present disclosure to provide improvements in relation to efficient and safe determination of what type of wound products to be used in relation to a wound of the patient, taking into account the current availability of wound products and at the same time matching present and previous indicative features of the patient's wound.

According to an aspect of the present disclosure, it is therefore provided a computer implemented method performed by a control unit arranged in communication with a database for selecting at least one wound product for a patient, wherein the method comprises the steps of receiving, at the control unit, a present first collection of data indicative of a wound of the patient, determining, using the control unit, a present first parameter for the wound based on the present first collection of data for the wound, acquiring, from the database using the control unit, a previously determined first parameter for the same wound of the patient being based on an earlier received collection of data indicative of the wound of the patient, determining, using the control unit, a difference between the present first parameter for the wound and the previously determined first parameter for the wound, acquiring, from the database using the control unit and based on the determined difference and the present first parameter for the wound, a second parameter for the wound, the second parameter being indicative of a treatment intervention for the wound, determining, using the control unit, a desired functional wound product property based on a combination of the present first and the second parameter for the wound, selecting, using the control unit, the at least one wound product of a plurality of different type of wound products to match the desired functional wound product property, and providing, from the control unit, information indicative of the selected at least one wound product.

The present disclosure is based upon the realization that it in some situations may be inefficient for the patient's healing process to only rely on the latest wound treatment recommendations for a specific type of wound for achieving an efficient wound treatment. Rather, the inventors have concluded that such treatment recommendations preferably should be seen as only one of a plurality of components in an automated matching process for selecting the most suitable wound product(s) for the patient's wound.

Accordingly, in line with the present disclosure an automated determination is made as to the progress of the patient's wound, where this progress in combination with suitable treatment intervention for the specific type of wound is used as an input for determining a desired functional wound product property. Thus, the “previously determined first parameter for the same wound of the patient” therefore corresponds to information previously collected about the same wound for the same patient.

Accordingly, advantages following the present disclosure may for example include the possibility to reduce the overall cost for the health system (for example being one of a hospital, a clinic or in relation to general home care), since the desired functional wound product property is in turn matched with e.g. currently available wound products at the health system. Thus, the health system must not necessary seek to find for them non-available wound products but may instead rely on for them available wound products, at the same time ensuring the most efficient treatment of the wound of the patient.

Further advantages following the present disclosure includes the possibility to identify if the wound product to be used with the patient's wound must necessarily comprise an antimicrobial component. As a consequence, in case e.g. the matching process indicates that no antimicrobial component is necessary, the indicative information of the selected at least one wound product may include a “recommendation” not to use an antimicrobial component. From an overall perspective, such a recommendation may reduce the general risk of an antimicrobial resistance.

Within the context of the present disclosure, the expression “wound product” should be interpreted broadly, including any form or means suitable for use in relation to treatment of a patient's wound. As such, a wound product may for example include a wound dressing, a bandage, a topical applicant, a treatment methodology in combination with a specific type of wound dressing, etc. Further, present or future, wound products are possible and within the scope of the present disclosure.

In a possible embodiment of the present disclosure, the step of selecting the at least one wound product of a plurality of different type of wound products comprises matching but not over-matching the desired functional wound product property. Over-matching the desired functional wound product property should within the context of the present disclosure be understood to signify a situation where the selected wound product(s) provides functions that are at present not necessary for treating the patient's wound. Such functions may however be “nice to have”, while in accordance to this embodiment is not absolute needed to treat the patient's wound.

Conversely, under-matching the desired functional wound product property should accordingly be understood to signify a situation where the wound product does not completely fulfill the desired functional wound product property for treatment of the patient's wound. However, in accordance to the present disclosure more than a single wound product may be selected for matching the desired functional wound product property for treatment of the patient's wound. In accordance to a non-limiting example, based on e.g. the availability of wound products it could for example be possible to select e.g. a sterile pad or compress in combination with a bandage to hold the sterile pad or compress in place, rather than only being given the recommendation to select a wound dressing that is self-adhesive.

Advantageously, it may thus in line with the present disclosure be possible to ensure that the patient is given the best possible treatment for his/her wound at the current state wound, while simultaneously ensuring that the caregiver (such as e.g. the physician, a nurse or any person assisting the patient) is given the opportunity to make use of the type of wound dressing that are readily available for use in relation to the specific patient and the specific wound. In line with the present disclosure, the expressions caregiver, physician, clinician, etc. are used interchangeably.

Preferably, the present first collection of data comprises information relating to at least one of a wound size, a tissue composition, an exudate amount, and a position of the wound. Such information is preferably collected using e.g. a camera arranged in communication with the control unit, where the control unit in turn may apply an image processing scheme for extracting e.g. the above listed data. The image processing scheme may in some embodiments include the previously determined first parameter for the same wound of the patient for ensuring that the image (or video) acquired by the camera is (sufficiently) normalized (e.g. equal conditions for image acquisition providing comparable image data). Such an implementation may possibly allow difference determination step to be improved, since a normalized comparison may allow of an in comparison higher quality determination as to the progress of the patent's wound.

Advantageously, the method according to the present disclosure may in some embodiments further comprise performing modelling of the present first collection of data of the wound and a previously stored collection of wound data for determining a present healing trajectory. That is, the control unit may form a model of the wound and its progress over time. The model of the wound, in its current and previous state, may in turn be used in a computer vision comparison-based process with e.g. a collection of corresponding wounds for other (non-related) patients. Such a collection of data may for example be stored in relation to the above-mentioned database.

Furthermore, it may in accordance to the present disclosure be possible to make use of the determined present healing trajectory for the wound for determining the desired functional wound product property. The healing trajectory may be positive or negative, where positive indicates a healing wound and negative indicate a worsen wound. Accordingly, the desired functional wound product property may in some embodiment be dependent on e.g. the gradient of the healing trajectory for the patient's wound; possibly resulting in a different selection of wound product(s) to be used for treatment of the patient's wound.

Additionally, in some embodiments it may be possible to acquire, from the database using the control unit, a previously determined healing trajectory, and to determine, using the control unit, a first trajectory difference by comparing the present healing trajectory and the previously determined healing trajectory. As such, it could in line with the present disclosure be possible to identify if the patient's wound is healing in line with what was previously expected, or if there is a deviation from e.g. how well the wound appeared at a previous check-up by the caregiver. Such information may further be used for determining the desired functional wound product property, and as a consequence in the selection of wound product(s) to be used for treatment of the patient's wound.

The first trajectory difference may additionally, in some embodiments, be compared to a predetermined threshold and/or range. That is, in case e.g. the first trajectory difference differs “too much” from what is expected (such as 5-20%), then this may be interpreted, by the control unit, as indicative of that the wound should be further investigated to ensure that the healing of the wound behaves as expected. Alternatively, or also, an unexpected deviation in regards to the first trajectory difference could be indicative of that the measurements are somewhat incorrect and should be made again. This information is preferably put forward, such as e.g. to the patient and/or the caregiver, for example as an alert.

In line with the present disclosure it may also be possible to apply a machine learning process to the present first collection of data of the wound and the previously stored collection of wound data. Such machine learning may for example be used in relation to determine the healing trajectory and/or for determining the first trajectory difference. Machine learning may also be used for determining the wound type.

A further advantage using machine learning is that the present first collection of data of the wound may be mapped not only to the previously stored collection of wound data, but also to other data relating to wounds for other non-related patients. Accordingly, the present first collection of data of the wound may be correlated to a large number of collections of data for determining status, healing, etc. for the present wound of the patient.

The machine learning process may for example be one of an unsupervised machine learning process or a supervised machine learning process. Using a supervised machine learning process may be initially advantageous (when in comparison a smaller amount of data is available), whereby the result of the machine learning process may be reviewed by a person, such as the caregiver, for determining if the outcome of the machine learning process is likely correct.

Different types of machine learning processes may be used, such as for example a machine learning process based on a convolutional neural network (CNN) or a recurrent neural network (RNN).

Turning back to the first trajectory difference, if the determined first trajectory difference is greater than the predetermined threshold, then the method may further comprise the step of updating the database to comprise the present first collection of data of the wound. Accordingly, in case the selected wound product or set of wound products applied to the wound result in an unexpected result, such as if the first trajectory difference is above/below a threshold or outside a predefined range, then it may be desirable to “feedback” this result to the database. It should further be noted that it also may be desirable to feedback the result in case the first trajectory difference is inside the predefined range, providing an indication of a correct selection of wound product(s) for treating the wound.

In one embodiment it may also or alternatively be possible to acquire, from the database using the control unit, a desired healing trajectory, and determining, using the control unit, a second trajectory difference by comparing the present healing trajectory and the previously determined healing trajectory. The second trajectory difference may in a similar manner as discussed above be used in determining the desired functional wound product property.

An advantage following such an implementation is that it may be possible to e.g. set a “treatment goal” for the patient, thus comparing an actual “healing trend” for the patient with a desired goal for the patient. The treatment goal may possibly be set dependent on specific factors for the patient, for the hospital, etc. The treatment goal may also be set based on a type of the patient, a type of the wound (e.g. a wound at a leg being different to a wound at an arm), etc.

As an example, in case the selected wound product or set of wound products applied to the wound results in an unexpected positive result for the wound of the patient, e.g. as to the healing rate, then it may be of high interest to ensure that this information can be used later by other patients having the same or similar type of wound. This concept may of course be applied the other way around, meaning that an unexpected negative result also is stored for allowing future/other patients to e.g. not select the same wound product or set of wound products for a similar wound. The machine learning process may also be applied for determining this unexpected result for the wound of the patient.

According to another aspect of the present disclosure, there is further provided a computer system adapted for selecting at least one wound product for a patient, the computer system comprising a server arranged in communication with a database, and a client device arranged in networked communication with the server, wherein the computer system is adapted to receive, at the server, a present first collection of data indicative of a wound of the patient, determine, using the server, a present first parameter for the wound based on the present first collection of data for the wound, acquire, from the database using the server, a previously determined first parameter for the same wound of the patient being based on an earlier received collection of data indicative of the wound of the patient, determine, using the server, a difference between the present first parameter for the wound and the previously determined first parameter for the wound, acquire, from the database using the server and based on the determined difference and the present first parameter for the wound, a second parameter for the wound, the second parameter being indicative of a treatment intervention for the wound, determine, using the server, a desired functional wound product property based on a combination of the present first and the second parameter for the wound, select, using the server, the at least one wound product of a plurality of different type of wound products to match the desired functional wound product property, provide, from the server to the client device, information indicative of the selected at least one wound product, and display, at the client device, the information indicative of the selected at least one wound product. This aspect of the present disclosure provides similar advantages as discussed above in relation to the previous aspects of the present disclosure.

In a possible embodiment of the present disclosure the client device is a mobile electronic device, such as e.g. at least one of a “dedicated electronic device”, a mobile phone, a tablet, etc. The client device may as an alternative be computer (e.g. laptop), for example provided with the above discussed camera for acquiring an image or video sequence of the wound of the patient. The client device may for example be arranged to be operated by the caregiver. Possibly, the client device may be adapted to collect or form a third-dimensional (3D) modelling of the wound of the patient, for example based on the collected images or video sequence.

In a preferred embodiment of the present disclosure the client device comprises a graphical user interface (GUI) adapted to provide the caregiver with an instruction for acquiring an image or video sequence of the wound of the patient or for otherwise inputting the present first collection of data indicative of a wound of the patient. The GUI may then, following the above discussed processing steps, be adapted to present the information indicative of the selected at least one wound product. The GUI may also be adapted to provide the caregiver with the above discussed information in case the healing process is behaving in an unexpected manner.

In one embodiment of the present disclosure the step of determining the desired functional wound product property may further be based on a geographical location of the patient. Accordingly, the recommendations given to e.g. the caregiver may be “localized”, for example ensuring that the recommendation provided follows local rules for what type of treatment that may be given to a patient.

In another embodiment of the present disclosure the patient may be selected to belong to at least one of a plurality of patient categories, and the step of determining the desired functional wound product property is further based on the selected category for the patient. Accordingly, it may in line with the present disclosure be possible to allow different patient categories to be treated slightly different, for example allowing different treatments to be provided to different age groups. Thus, for example children may be given a slightly different treatment as compared to adults.

According to a still further aspect of the present disclosure there is provided a computer program product comprising a non-transitory computer readable medium having stored thereon computer program means for operating a computer system adapted for selecting at least one wound product for a patient, the computer system comprising a server arranged in communication with a database, and a client device arranged in networked communication with the server, wherein the computer program product comprises code for receiving, at the server, a present first collection of data indicative of a wound of the patient, code for determining, using the server, a present first parameter for the wound based on the present first collection of data for the wound, code for acquiring, from the database using the server, a previously determined first parameter for the same wound of the patient being based on an earlier received collection of data indicative of the wound of the patient, code for determining, using the server, a difference between the present first parameter for the wound and the previously determined first parameter for the wound, code for acquiring, from the database using the server and based on the determined difference and the present first parameter for the wound, a second parameter for the wound, the second parameter being indicative of a treatment intervention for the wound, code for determining, using the server, a desired functional wound product property based on a combination of the present first and the second parameter for the wound, code for selecting, using the server, the at least one wound product of a plurality of different type of wound products to match the desired functional wound product property, code for providing, from the server to the client device, information indicative of the selected at least one wound product, and code for displaying, at the client device, the information indicative of the selected at least one wound product. Also this aspect of the present disclosure provides similar advantages as discussed above in relation to the previous aspects of the present disclosure.

Further features of, and advantages with, the present disclosure will become apparent when studying the appended claims and the following description. The skilled addressee realizes that different features of the present disclosure may be combined to create embodiments other than those described in the following, without departing from the scope of the present disclosure.

### BRIEF DESCRIPTION OF THE DRAWINGS

The various aspects of the present disclosure, including its particular features and advantages, will be readily understood from the following detailed description and the accompanying drawings, in which:

FIG. 1 conceptually shows a computer system according to a currently preferred embodiment of the present disclosure adapted for selecting a wound product for a patient,

FIG. 2 discloses a possible client device comprising a graphical user interface for use in relation to the present wound product selection,

FIGS. 3A-3C shows an exemplary wound in different stages of healing,

FIGS. 4A-4C shows tables presenting functional wound product property and matching with a plurality of different wound products for the different wound stages as shown in FIGS. 3A-3C,

FIGS. 5A and 5B presents different approaches for treating the wound as shown in FIG. 3B, and

FIG. 6 is a flow chart illustrating the steps of performing the method according to a currently preferred embodiment of the present disclosure.

### DETAILED DESCRIPTION

The present disclosure will now be described more fully hereinafter with reference to the accompanying drawings, in which currently preferred embodiments of the present disclosure are shown. The present disclosure may, however, be embodied in many different forms and should not be construed as limited to the embodiments set forth herein; rather, these embodiments are provided for thoroughness and completeness, and fully convey the scope of the present disclosure to the skilled person. Like reference characters refer to like elements throughout.

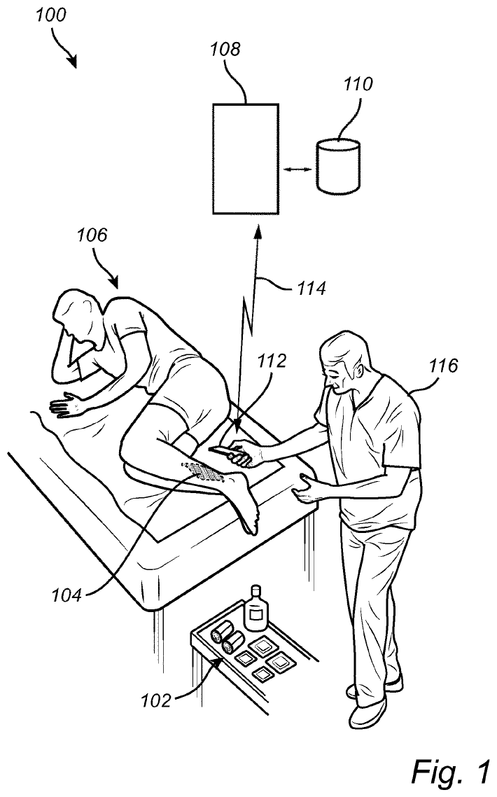

Turning now to the drawings and to FIG. 1 in particular, there is conceptually illustrated a computer system 100 adapted for selecting one or a set of wound products 102 for a wound 104 of a patient 106.

The computer system comprises a server 108 arranged in communication with a database 110, and a client device 112 arranged in networked communication, such as using the Internet 114, with the server 108. In FIG. 1, the client device 112 is operated by a caregiver 116; however, any user, such as for example any form of caregiver, may be allowed to operate the client device 112.

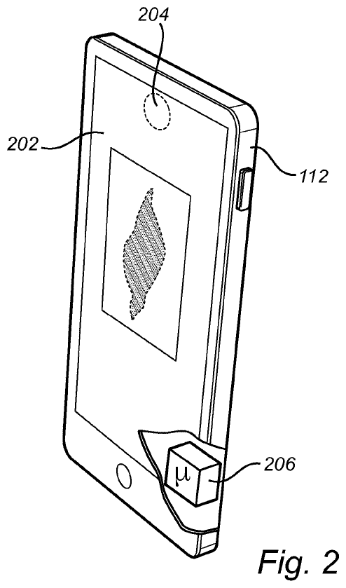

The client device 112, as further detailed in FIG. 2 and illustrated as a mobile phone, comprises a graphical user interface (GUI) 202 and a camera 204. The client device 112 further comprises a control unit 206. The GUI 202 is preferably adapted to present instructions and information to e.g. the caregiver 116, such as for acquiring images of the wound 104 of the patient 106 using the camera 204 and for displaying information as to one or a set of wound products 102 to be used for treating the wound 104.

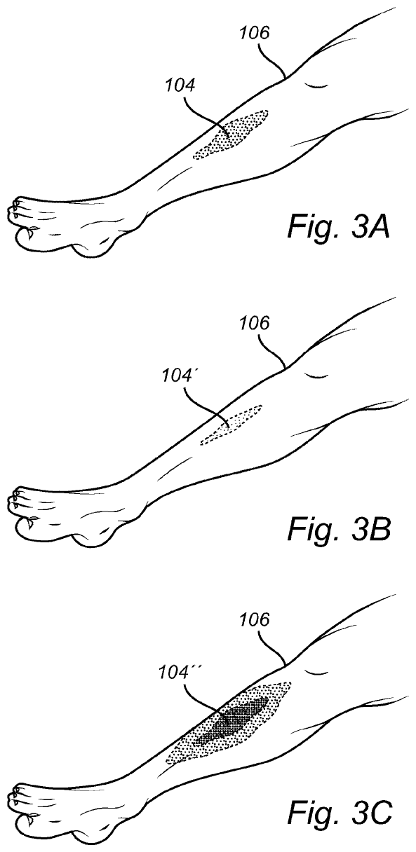

FIGS. 3A-3C shows an exemplary wound in different stages of healing. Specifically, in FIG. 3A there is exemplified the wound 104 at a time when the patient 106 is meeting the caregiver 116 for the first time, such as at a clinic or hospital. At this time, the caregiver 116 will use his/her client device 112 for collecting data in relation/about the wound 104.

In line with an exemplary embodiment of the present disclosure, the GUI 202 displayed at the client device 102 will present a request for the caregiver 116 to acquire an image of the wound 104. The control unit 206 will then perform an image processing scheme for determining a present first collection of data indicative of the wound 104. The image processing scheme may for example generate data indicative of a height/width/depth/surface area of the wound 104, as well as a color of the wound 104. Other data may be possible and within the scope of the present disclosure. The caregiver 116 may also (or alternatively) input corresponding data, and additional data such as a smell/malodor of the wound 104.

The present first collection of data indicative of the wound 104 is then processed for determining a present first parameter for the wound 104. While the image may be seen as comprising the present first collection of data, the present first parameter for the wound 104 may be interpreted as indicative features for the wound 104, indicative of the status of the wound 104.

The present first parameter for the wound 104 is subsequently transmitted to the server 108 (which may be local or remotely arranged) for storage at the database 110. It may also be possible to store the present first collection of data indicative of the wound 104 locally in a database at a memory element comprised with the client device 112. The server 108 and/or the client device 112 may (separate or in combination), based on the present first collection of data indicative of the wound 104, determine one or a set of wound products for use to treat the wound 104.

When the patient 106 again meets the caregiver 116 (e.g. being the same or another caregiver), for example at a scheduled checkup a week later, the previously used one or a set of wound products are at least partly removed. The wound 104′ as shown in FIG. 3B visually appears to have improved since the last visit. At this stage, the caregiver 116 (once again) acquires an image indicative of a present first collection of data indicative of the wound 104′ of the patient 106. The control unit 206 again processes the present first collection of data indicative of the wound 104′ for determining the present first parameter for the wound 104′.

The control unit 206 may for example at this stage acquire the first parameter for the same wound of the patient 106, as was previously determined at the first visit at the caregiver 116. The control unit 206 will at this stage determine a difference between the present first parameter for the wound 104′ with the previous first parameter for the wound 104.

The present first parameter for the wound 104′ and the determined difference between the wounds 104, 104′ at its different stages is then used as two inputs to the database 110, where the database 110 typically stores a plurality of treatment intervention for different type of wounds. In a possible embodiment of the present disclosure the server 108 takes the two inputs for matching with the treatment intervention for the different types of wounds, and then selects at least one treatment intervention (defined as a second parameter) determined to correlate with the present first parameter for the wound 104′ and the determined difference between the wound 104, 104′.

The server 108 and/or the control unit 206 then determines one or a plurality of desired functional wound product properties based on a combination of the present first and the second parameter for the wound. The one or a plurality of desired functional wound product properties will accordingly be specifically targeted for the present status of the wound 104′, in combination with how the wound 104′ has progressed (since e.g. the above-mentioned week ago) and what type of suitable treatment intervention that should be applied in this specific situation.

It may then be possible to match the one or a plurality of desired functional wound product properties with different types of wound products, possibly limited to a currently available set of wound products 102.

That is, from a general perspective different type of wound products may have different “wound healing properties”, and the most suitable selection is in line with the present disclosure selected by taking into account all of current status, progress, and generalized recommendation for the specific type of wound 104′.

At this state, the GUI 202 of the client device 112 may display information indicative of the selected one or a set of wound products 102 for use to treat the wound 104′. The information may comprise instructions on how to dress the wound 104′, in what order to apply e.g. the set of wound products 102, etc.

Within the scope of the present disclosure, the GUI may also include a virtual reality or augmented reality component for example used for displaying the information indicative of the selected one or a set of wound products 102 for use to treat the wound 104′. As such, the information provided to e.g. the caregiver may use the virtual reality or augmented reality component for “correctly” providing e.g. the selected one or a set of wound products 102 for use to treat the wound 104′, and/or for visualizing e.g. an expected progress of the wound 104′ at or in a relation to the position of the wound 104′.

An alternative situation is shown in FIG. 3C, where instead the wound 104″ visually appears to have worsened since the last visit. That is, in FIG. 3C the wound 104″ appears to have increased in both surface area and depth. The caregiver 116 once again acquires an image of the wound 104″, and a present first parameter for the wound 104″ as well as a difference between the wounds 104, 104″ is determined, in a corresponding manner as discussed in relation to FIG. 3B. This information is in a corresponding manner used for determining a desired functional wound product property for the wound 104″.

In a possible embodiment of the present disclosure, the desired functional wound product property for the wound 104″ differs from the desired functional wound product property for the wound 104′ as shown in FIG. 3B. Correspondingly, the one or a set of wound products 102 for use to treat the wound 104″ may possibly differ from the one or a set of wound products 102 for use to treat the wound 104′.

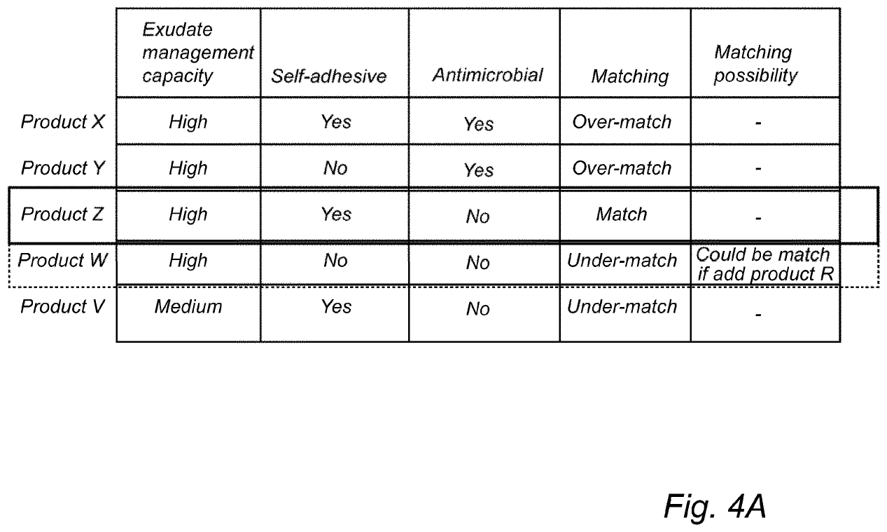

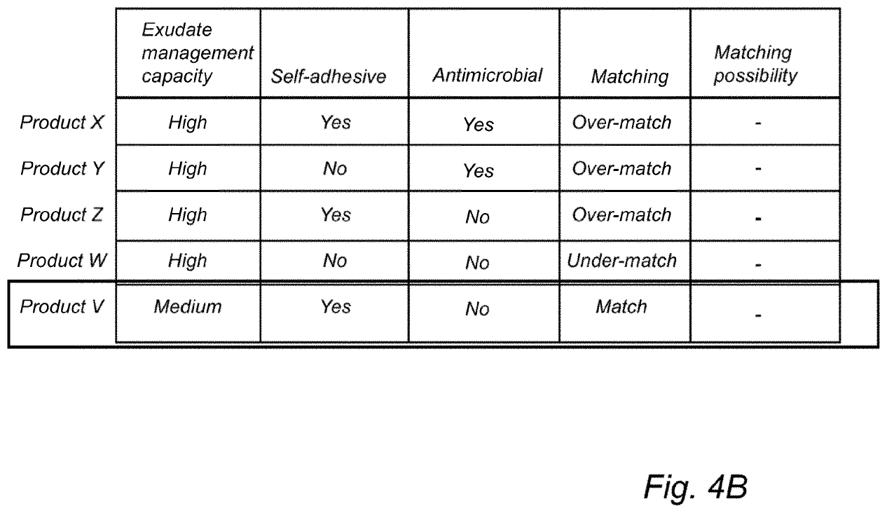

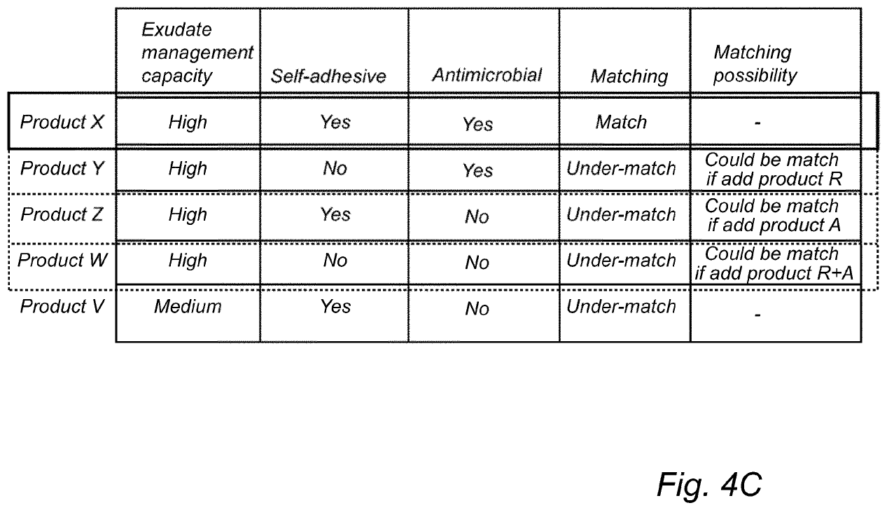

FIGS. 4A-4C shows three different tables presenting functional wound product property and matching with a plurality of different wound products for the different wound stages as shown in FIGS. 3A-3C, respectively.

The table in FIG. 4A relates to the wound 104. It has in this case been determined that the wound 104 requires a high exudate management capacity. No signs of infection is however present. As shown in FIG. 4A, an exemplary product “Z” has been matched as optimal for the wound 104. However, an exemplary product “W” has been identified as only being a “partial match” that needs to be complemented with a retention product (indicated as exemplary product “R”). On the other hand, exemplary products “X” and “Y” are determined to be over-matching the wound 104, whereas an exemplary product “V” is under-matched and not possible to be complemented with a further wound product to achieve the desired functionality.

In regards to the wound 104′, as shown in FIG. 3B and with further reference to FIG. 4B, the wound is progressing according to an expected healing trajectory. An area/volume of the wound 104′ is contracting and exudate management capacity requirements are therefore determined to be moderate (as compared to FIG. 3A, 4A). Again, no signs of infection are indicated. Based on the assessment, the exemplary product “V” is determined to be an optimal match for the wound 104′. However, exemplary products “X”, “Y”, “Z”, “W” are determined to be over-matched (in line with the discussion above).

In regards to the wound 104″, as shown in FIG. 3B and with further reference to FIG. 4C, the wound 104″ is (unfortunately) not progressing according to an expected healing trajectory. A localized infection has in this case been identified, the wound size is slightly larger, and the exudate amount is constant (as compared to FIG. 3A). The exemplary product “X” is here determined to provide an optimal match to the wound 104″. However, products “Y”, “Z” and “W” are partial matches and needs to be complemented. The complement is for example exemplary product “R” for providing retention, or exemplary product “A” for providing an antimicrobial effect.

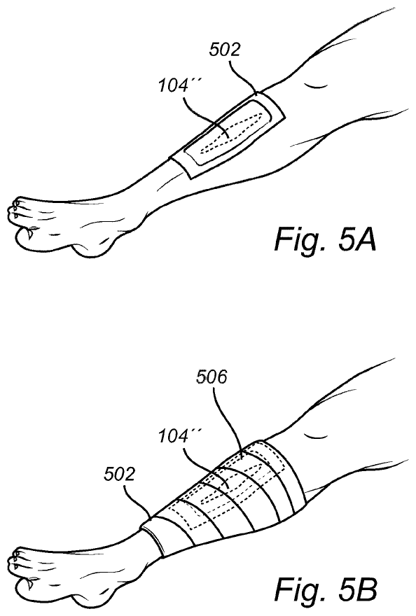

Turning now to FIGS. 5A and 5B, provided in line with the above examples and resenting different approaches to treating the wound 104″ as illustrated in FIG. 3C. As exemplified, in FIG. 5A the wound 104″ is treated using a wound product 102 (corresponding to exemplary wound product “X” as shown in FIG. 4C), specifically a self-adhesive wound dressing 502.

Conversely, in FIG. 5B there is presented another approach for treating the same wound 104″ as shown in FIG. 3C, however using a different type of wound dressing 102. Specifically, in FIG. 5B a sterile pad 504 (corresponding to exemplary wound product “Y” as shown in FIG. 4C) is used in combination with a bandage 506 (corresponding to exemplary wound product “R” as shown in FIG. 4C) to hold the sterile pad 504 in place. In both of FIGS. 5A and 5B the treatment of the wound 104″ is optimized for ensuring a desirable treatment of the wound 104″, however not limited to only a single approach for treating the wound.

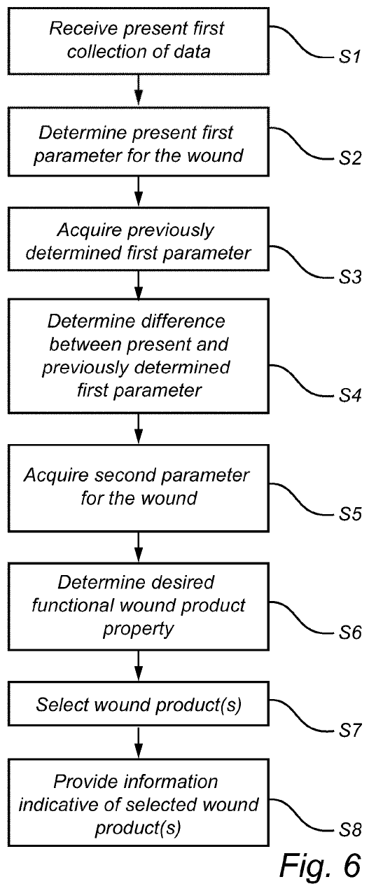

In summary and with further reference to FIG. 6, the present disclosure relates to a computer implemented method performed by a control unit 206 arranged in communication with a database 110 for selecting at least one wound product 102 for a patient 106, wherein the method comprises the steps of receiving, S1, at the control unit 206, a present first collection of data indicative of a wound 104′, 104″ of the patient 106, determining, S2, using the control unit 206, a present first parameter for the wound 104′, 104″ based on the present first collection of data for the wound 104′, 104″, acquiring, S3, from the database 110 using the control unit 206, a previously determined first parameter for the same wound 104 (but possibly at a different state) of the patient 106.

The method further comprises determining, S4, using the control unit 206, a difference between the present first parameter for the wound 104′, 104″ and the previously determined first parameter for the wound 104, acquiring, S5, from the database 110 using the control unit 206 and based on the determined difference and the present first parameter for the wound 104′, 104″, a second parameter for the wound 104′, 104″, the second parameter being indicative of a treatment intervention for the wound 104′, 104″, determining, S6, using the control unit 206, a desired functional wound product property based on a combination of the present first and the second parameter for the wound 104′, 104″, selecting, S7, using the control unit 206, the at least one wound product 102 of a plurality of different type of wound products 102 to match the desired functional wound product property, and providing, S8, from the control unit 206, information indicative of the selected at least one wound product 102.

The present disclosure is based upon the realization that it in some situations may be inefficient for the patient's healing process to only rely on the latest wound treatment recommendations for a specific type of wound for achieving an efficient wound treatment. Rather, the inventors have concluded that such treatment recommendations preferably should only be seen as one of a plurality of components in an automated matching process for selecting the most suitable wound product(s) 102 for the patient's wound 104′, 104″.

The control functionality of the present disclosure may be implemented using existing computer processors, or by a special purpose computer processor for an appropriate system, incorporated for this or another purpose, or by a hardwire system. Embodiments within the scope of the present disclosure include program products comprising machine-readable medium for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor.

Although the figures may show a sequence, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps. Additionally, even though the present disclosure has been described with reference to specific exemplifying embodiments thereof, many different alterations, modifications and the like will become apparent for those skilled in the art.

In addition, variations to the disclosed embodiments can be understood and effected by the skilled addressee in practicing the present disclosure, from a study of the drawings, the disclosure, and the appended claims. Furthermore, in the claims, the word “comprising” does not exclude other elements or steps, and the indefinite article “a” or “an” does not exclude a plurality.

1. A computer implemented method performed by a control unit arranged in communication with a database for selecting at least one wound product for a patient, wherein the method comprises:
receiving, at the control unit, a present first collection of data indicative of a wound of the patient,
determining, using the control unit, a present first parameter for the wound based on the present first collection of data for the wound,
acquiring, from the database using the control unit, a previously determined first parameter for the same wound of the patient being based on an earlier received collection of data indicative of the wound of the patient,
determining, using the control unit, a difference between the present first parameter for the wound and the previously determined first parameter for the wound,
acquiring, from the database using the control unit and based on the determined difference and the present first parameter for the wound, a second parameter for the wound, the second parameter being indicative of a treatment intervention for the wound,
determining, using the control unit, a desired functional wound product property based on a combination of the present first and the second parameter for the wound,
selecting, using the control unit, the at least one wound product of a plurality of different type of wound products to match the desired functional wound product property, and
providing, from the control unit, information indicative of the selected at least one wound product.

2. The method according to claim 1, wherein the step of selecting the at least one wound product of a plurality of different type of wound products comprises matching but not over-matching the desired functional wound product property.

3. The method according to claim 1, wherein the present first collection of data comprises information relating to at least one of a wound size, a tissue composition, an exudate amount, and a position of the wound.

4. The method according to claim 1, further comprising:
performing, using the control unit, a modelling of the present first collection of data of the wound and a previously stored collection of wound data for determining a present healing trajectory.

5. The method according to claim 4, wherein the desired functional wound product property is further determined based on the determined present healing trajectory.

6. The method according to claim 1, wherein the previously stored collection of wound data is acquired from the database.

7. The method according to claim 4, further comprising the steps of:
acquiring, from the database using the control unit, a previously determined healing trajectory, and
determining, using the control unit, a first trajectory difference by comparing the present healing trajectory and the previously determined healing trajectory.

8. The method according to claim 7, wherein the desired functional wound product property is further determined based on the determined first trajectory difference.

9. The method according to claim 7, further comprising determining if the first trajectory difference is greater than a predetermined threshold.

10. The method according to claim 7, further comprising:
forming an alert if the determined first trajectory difference is greater than the predetermined threshold.

11. The method according to claim 4, further comprising:
acquiring, from the database using the control unit, a desired healing trajectory, and
determining, using the control unit, a second trajectory difference by comparing the present healing trajectory and the previously determined healing trajectory.

12. The method according to claim 11, wherein the desired functional wound product property is further determined based on the determined second trajectory difference.

13. The method according to claim 1, wherein the present first collection of data comprises at least one of an image and a video sequence of an area of the wound of the patient.

14. The method according to claim 13, wherein the video sequence comprises a third-dimensional (3D) modelling of the wound of the patient.

15. The method according to claim 4, wherein the modelling of the present first collection of data of the wound and the previously stored collection of wound data comprises applying a machine learning process.

16-18. (canceled)

19. The method according to claim 1, wherein the step of determining the desired functional wound product property is further based on a geographical location of the patient.

20. The method according to claim 1, wherein the patient is selected to belong to at least one of a plurality of patient categories, and the step of determining the desired functional wound product property is further based on the selected category for the patient.

21. The method according to claim 4, wherein if the determined trajectory difference is greater than the predetermined threshold the method further comprising:
updating the database to comprise the present first collection of data of the wound.

22. A computer system adapted for selecting at least one wound product for a patient, the computer system comprising:
a server arranged in communication with a database, and
a client device arranged in networked communication with the server, wherein the computer system is adapted to:
receive, at the server, a present first collection of data indicative of a wound of the patient,
determine, using the server, a present first parameter for the wound based on the present first collection of data for the wound,
acquire, from the database using the server, a previously determined first parameter for the same wound of the patient being based on an earlier received collection of data indicative of the wound of the patient,
determine, using the server, a difference between the present first parameter for the wound and the previously determined first parameter for the wound,
acquire, from the database using the server and based on the determined difference and the present first parameter for the wound, a second parameter for the wound, the second parameter being indicative of a treatment intervention for the wound,
determine, using the server, a desired functional wound product property based on a combination of the present first and the second parameter for the wound,
select, using the server, the at least one wound product of a plurality of different type of wound products to match the desired functional wound product property,
provide, from the server to the client device, information indicative of the selected at least one wound product, and
display, at the client device, the information indicative of the selected at least one wound product.

23. A computer program product comprising a non-transitory computer readable medium having stored thereon computer program means for operating a computer system adapted for selecting at least one wound product for a patient, the computer system comprising:
a server arranged in communication with a database, and
a client device arranged in networked communication with the server, wherein the computer program product comprises:
code for receiving, at the server, a present first collection of data indicative of a wound of the patient,
code for determining, using the server, a present first parameter for the wound based on the present first collection of data for the wound,
code for acquiring, from the database using the server, a previously determined first parameter for the same wound of the patient being based on an earlier received collection of data indicative of the wound of the patient,
code for determining, using the server, a difference between the present first parameter for the wound and the previously determined first parameter for the wound,
code for acquiring, from the database using the server and based on the determined difference and the present first parameter for the wound, a second parameter for the wound, the second parameter being indicative of a treatment intervention for the wound,
code for determining, using the server, a desired functional wound product property based on a combination of the present first and the second parameter for the wound,
code for selecting, using the server, the at least one wound product of a plurality of different type of wound products to match the desired functional wound product property,
code for providing, from the server to the client device, information indicative of the selected at least one wound product, and
code for displaying, at the client device, the information indicative of the selected at least one wound product.
